(19) United States
(12) Patent Application Publication
(10) Pub. No.: US 2026/0235575 A1
(43) Pub. Date: Aug. 13, 2026

(54) ON THE GO IN-SITU SOIL GAS SAMPLING

(71) Applicant: Deere & Company, Moline, IL (US)

(72) Inventors: Timothy A. Wilcox, Cissna Park, IL (US); Blake Hedrick, Champaign, IL (US); Lance S. Conway, New Haven, MO (US); BRADLEY W. VAN DE WOESTYNE, West Des Moines, IA (US)

(21) Appl. No.: 19/051,329

(22) Filed: Feb. 12, 2025

(51) Int. Cl. G01N 33/24; A01B 63/00

(52) U.S. Cl. CPC G01N 33/245; A01B 63/008

(57) ABSTRACT

A soil gas sampling implement includes a soil gas sensor system configured to sample soil gas at multiple depths and configured to generate a soil gas content signal corresponding to a concentration of one or more soil gas constituents of the sampled soil gas. The soil gas sensor system includes at least one sensing probe configured to collect soil gas samples. A position sensor may be configured to generate a position signal corresponding to a geographic location of the implement within the field. A controller may be functionally linked with the soil gas sensor system and the position sensor for receiving the soil gas content signal and the position signal, the controller being configured to send a command signal to the soil gas sensor system to adjust a sensing depth at which the soil gas is sampled as the implement traverses the field.

### BACKGROUND OF THE DISCLOSURE

### 1. Field of the Disclosure

The present disclosure generally relates to systems for the sampling of soil gas to measure the efflux of one or more soil gas constituents, especially carbon dioxide (CO₂), from the soil.

### 2. Description of the Prior Art

Traditional in-field soil CO₂ measurements are made from static sensors on the soil surface, or through probes inserted at varying depths within the soil profile. Surface CO₂ sensors estimate soil CO₂ efflux, which has been found to vary with cropping system and soil type. These types of measurements are limited in space, and do not provide an understanding of within-field spatial variability.

Soil CO₂ efflux is the rate at which soil exchanges carbon dioxide with the atmosphere. It's also known as soil respiration. Soil CO₂ efflux is a major contributor to the exchange of carbon dioxide on land, second only to terrestrial photosynthesis. Soil CO₂ efflux is the result of two processes: (1) soil CO₂ production, which is the result of autotrophic respiration by roots and heterotrophic respiration by microbes decomposing organic matter; and (2) transport to the atmosphere, which is the movement of CO₂ from the soil to the atmosphere. Soil CO₂ efflux is affected by temperature and precipitation. Soil temperature changes are a major driver of seasonal CO₂ efflux. Precipitation can also increase soil CO₂ efflux by displacing soil gas or increasing microbial activity.

In the lab, soil CO₂ respiration rates are derived in controlled conditions. These measures have been used as an indicator of microbial activity and have also been found to relate to economically optimum nitrogen fertilizer application rates. This relationship is attributed to nitrogen mineralization induced by microbially activity.

An on-the-go system for detecting the concentration of gases in soil has been proposed in Herring, U.S. Patent Application Publ. No. 2023/0085819, the details of which are incorporated herein by reference.

There is a continuing need for improvement of such on-the-go systems to improve the accuracy of the soil CO₂ concentration sensors reading of CO₂ concentration, as well as the accuracy of conversion of those concentration readings to soil CO₂ efflux.

### SUMMARY OF THE DISCLOSURE

The present disclosure provides improved apparatus and methods of soil gas extraction to increase the accuracy and interpretability of the CO₂ concentration measurements. Additionally, improved methods are provided for estimating the CO₂ efflux from the soil based on the CO₂ concentration measurements and other crop management information, both from sensed data and from publicly available soil and weather information.

In one embodiment a soil gas sampling implement includes a soil gas sensor system configured to sample soil gas at multiple depths from soil of a field being traversed by the implement. The soil gas sensor system is further configured to generate a soil gas content signal corresponding to a concentration of one or more soil gas constituents of the soil gas sampled by the soil gas sensor system, the soil gas sensor system including at least one sensing probe configured to collect soil gas samples. A position sensor may be configured to generate a position signal corresponding to a geographic location of the implement within the field. A controller may be functionally linked with the soil gas sensor system and the position sensor for receiving the soil gas content signal and the position signal, the controller being configured to send a command signal to the soil gas sensor system to adjust a sensing depth at which the soil gas is sampled as the implement traverses the field.

In another embodiment a method of sampling soil gas may include: sampling soil gas from soil of a field with at least one sensing probe of an implement as the field is being traversed by the implement; generating a soil gas concentration signal corresponding to a concentration of one or more soil gas constituents of the soil gas sampled by the at least one sensing probe; detecting a geographic location of the implement within the field using a position sensor associated with the implement and configured to generate a position signal corresponding to a geographic location of the implement within the field; receiving the soil gas concentration signal and the position signal with a controller; and generating a command signal with the controller and thereby adjusting a sensing depth at which the soil gas is sampled as the implement traverses the field.

Numerous objects, features and advantages of the embodiments set forth herein will be readily apparent to those skilled in the art upon reading of the following disclosure when taken in conjunction with the accompanying drawings.

### BRIEF DESCRIPTION OF THE DRAWINGS

FIG. 1 is a schematic side elevation view of one embodiment of a soil gas sampling implement towed by a tractor.

FIG. 2 illustrates the soil gas sampling implement of FIG. 1 adjusted to a deepest sampling depth.

FIG. 3 illustrates the soil gas sampling implement of FIG. 1 adjusted to an intermediate sampling depth.

FIG. 4 illustrates the soil gas sampling implement of FIG. 1 adjusted to a shallow sampling depth.

FIG. 5 is a schematic depiction of the soil gas sampling system of the implement of FIG. 1.

FIG. 6 is a schematic depiction of a control system of the soil gas sampling implement.

FIG. 7 is a schematic side elevation view of another embodiment of a soil gas sampling implement towed by a tractor where multiple probes are provided at different sensing depths.

FIG. 8 is a schematic depiction of the soil gas sampling system of the implement of FIG. 7.

FIG. 9 is a schematic side elevation view of another embodiment of a soil gas sampling implement towed by a tractor wherein multiple sensing probes are arranged in a line one behind the other.

FIG. 10 is a graphical representation of a map generated by the controller representing soil gas constituent concentrations throughout the geographic area of a field.

FIG. 11 is a graphical representation of the relationship between the economically optimum nitrogen fertilizer amount to be added to a field as a function of the biological activity of the soil as indicated by CO₂ efflux measurements.

FIG. 12 is a graphical representation of the manner in which CO₂ efflux data from static sensors may be used in the analysis of the CO₂ efflux data from the on-the-go sensors.

FIG. 13 is a flow chart depicting how the controller may analyze and correlate other available data with the CO₂ efflux data to make decisions about nitrogen application to the field.

### DETAILED DESCRIPTION

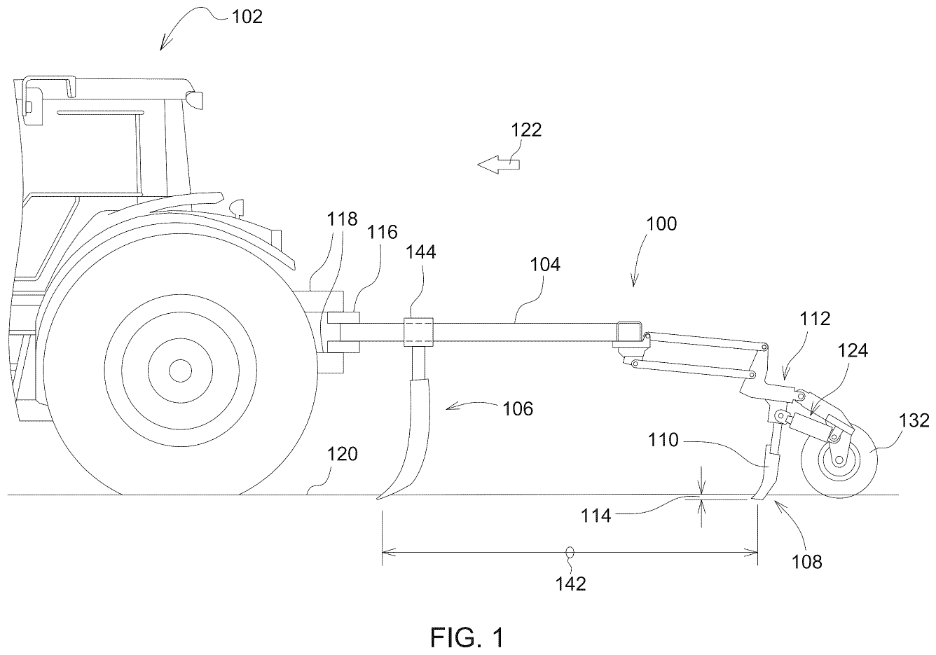

FIG. 1 schematically illustrates a soil gas sampling implement 100 towed by a tractor 102. The implement 100 includes an implement frame 104 which carries a soil disrupting tool 106 and a soil gas sensor system 108. The soil disrupting tool 106 may be any conventional tillage tool, such as a moldboard, chisel, disc, plow or the like.

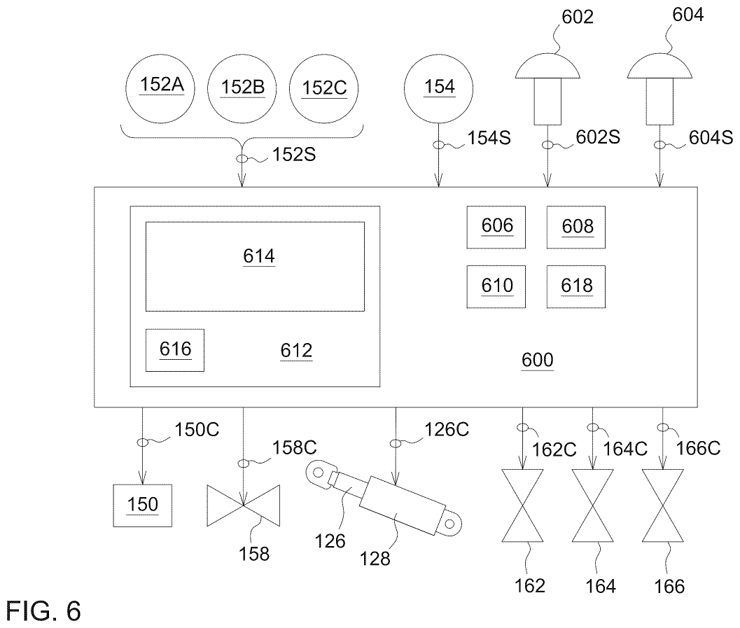

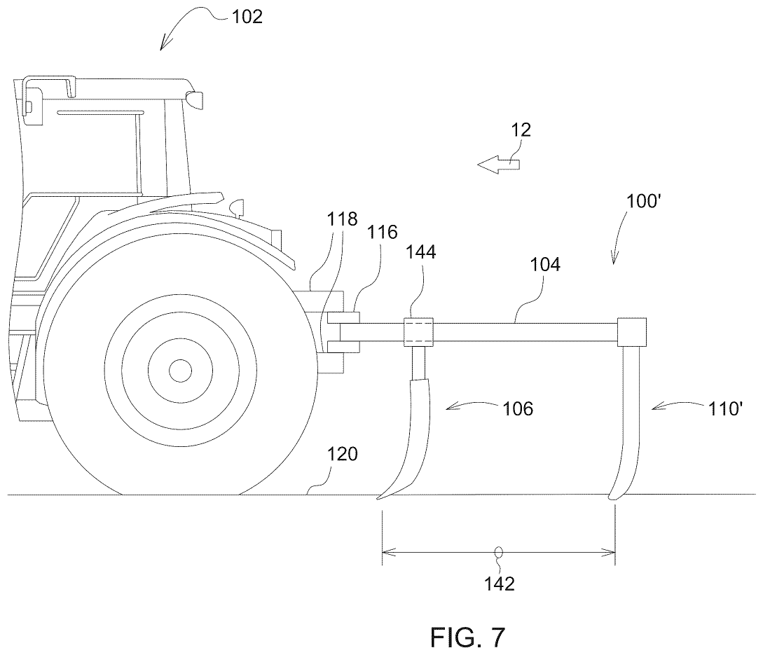

The soil gas sensor system 108 is configured to sample soil gas at multiple depths from soil of a field being traversed by the implement 100. The soil gas sensor system 108 is configured to generate a soil gas content signal 152S (see FIG. 6) corresponding to a concentration of one or more soil gas constituents of the soil gas sampled by the soil gas sensor system 108. The soil gas sensor system 108 includes at least one sensing probe 110 configured to collect soil gas samples. The soil disrupting tool 106 is supported from the implement frame 104 in front of the at least one sensing probe 110 with respect to a forward direction of travel 122.

The implement 100 may include a tongue 116 at its forward end, which tongue 116 may be connected to a hitch 118 of the tractor 102. The hitch 118 may for example be a conventional 3-point or 4-point hitch assembly. The entire implement 100 may be raised off the ground surface 120 or lowered to the ground surface 120 by raising and lowering the hitch 118.

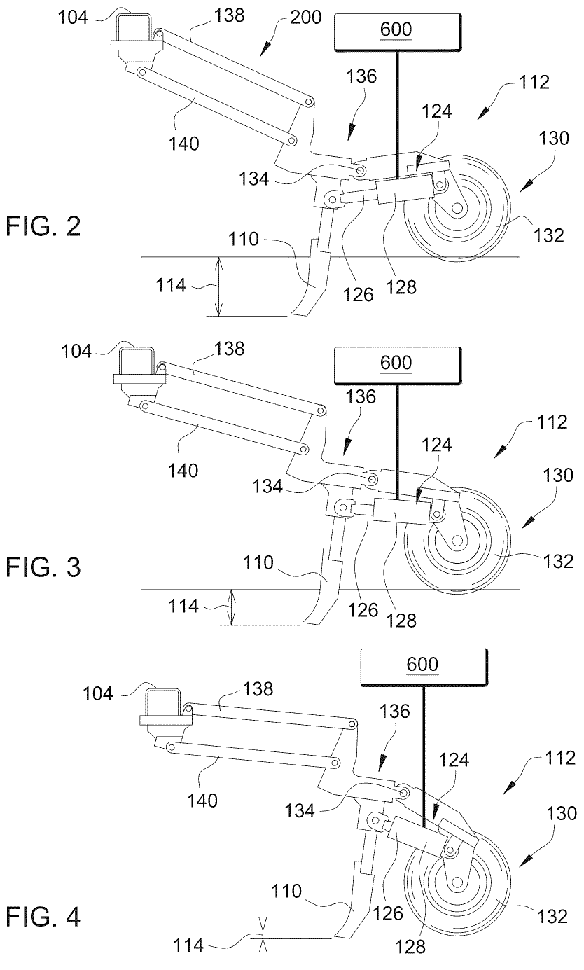

In the embodiment of FIGS. 1-4, the at least one sensing probe 110 is supported by a depth adjustment assembly 112 such that a sensing depth 114 of the at least one sensing probe 110 within the soil is adjustable. The operation of the depth adjustment assembly 112 is best shown in FIGS. 2-4 which illustrate the at least one sensing probe 110 at a greatest depth in FIG. 2, an intermediate depth in FIG. 3 and a shallow depth in FIG. 4.

The depth adjustment assembly 112 includes an actuator 124 including a piston 126 and cylinder 128. Different extensions of the piston 126 relative to the cylinder 128 define the different depths of the at least one sensing probe 110 seen in FIGS. 2-4. In the illustrated example, FIG. 2 shows a full stroke position of the piston 126, FIG. 3 shows a nominal stroke position of the piston 126, and FIG. 4 shows a closed position of the piston 126. That is, in the full stroke position, the piston 126 is fully extended from the cylinder 128; in the closed position, the piston 126 is fully retracted into the cylinder 128; and in the nominal stroke position, the piston 126 is partially extended from the cylinder 128 in-between the full stroke position and the closed position. It should be noted that the nominal stroke position can be adjusted as desired or needed to position the piston 126 at any point between fully extended and closed. That is, the nominal stroke position can be halfway or at fifty-percent (50%) of full extension or at some other percentage or level of extension. Similarly, the full stroke position of the piston 126 in some examples can be less than a one-hundred percent (100%) operationally extended position from the cylinder 128. For example, the extended positions of the piston 126, namely the full stroke position and the nominal stroke position can be adjusted, such as based on the configuration and/or depth requirements for the at least one sensing probe 110. As such, the positions of the piston 126 can be defined to differently vary the ground penetration depth level of the at least one sensing probe 110.

In some examples, the cylinder 128 is a two-stage cylinder having three operational positions or that mechanically defines the full stroke position and the nominal stroke position of the piston. That is, the structural and/or operational configuration of the cylinder 128 sets or defines the operational positions of the piston 126, namely the full stroke position, the nominal stroke position of the piston 126, and the closed position of the piston 126. It should be noted that variations and modifications are contemplated. For example, the cylinder 128 is various examples can be any multi-position or multi-stage cylinder 128, such as having more or less than three positions. That is, in some examples, the cylinder 128 has two positions or stages (e.g., fully extended and fully retracted), four positions or stages, etc. In some examples, the cylinder 128 does not have a fixed number of stages or positions but is a “smart” cylinder that is operable to define one or more positions or stages between the full stroke position and the nominal stroke position. In some examples, the actuator is a position controlled electrical actuator having a continuously variable piston length (e.g., infinitely variable). That is, any length of actuation of piston 126 can be provided instead of mechanical stepwise or incremental actuation lengths.

As can be seen in FIGS. 2-4, the piston 126 is operable to change sensing depth 114 of the at least one sensing probe 110 relative to a trailing end 130 of the implement 100, namely a trailing wheel 132 of the implement 100, by movement at a pivot point 134 of an arm 136. That is, a sensing depth 114 of the at least one sensing probe 110 is changed by the movement of the piston 126 to thereby adjust a relative height of the at least one sensing probe 110 and resulting in changing a penetration depth 114 of the at least one sensing probe 110 resulting by pivoting movement or rotation of the arm 136 at the pivot point 134. The pivot point 134 in various examples is configured or provided as any type or kind of pivoting or rotating member (e.g., a pivoting or rotating pin). As such, in various examples, rotation or pivoting of the arm 136 about the pivot point 134 causes a change in a height of the at least one sensing probe 110 relative to the other components (e.g., the wheels 132).

As schematically shown in FIG. 1, the soil disrupting tool 106 is spaced from the at least one sensing probe 110 by a distance 142 so as to create a time delay between the disruption of the soil by the soil disrupting tool 106 and sampling of the soil gas by the at least one sensing probe 110. The distance 142 may be in a range of from 1 to 12 feet in the travel 122. The soil disrupting tool 106 is shown as being adjustably mounted on the implement frame 104 by an adjustable mount 144. In general, one or both of the soil disrupting tool 105 and the at least one sensing probe 110 may be adjustably mounted relative to the implement frame 104 so that the distance 142 between the soil disrupting tool 106 and the sensing probe 110 is adjustable.

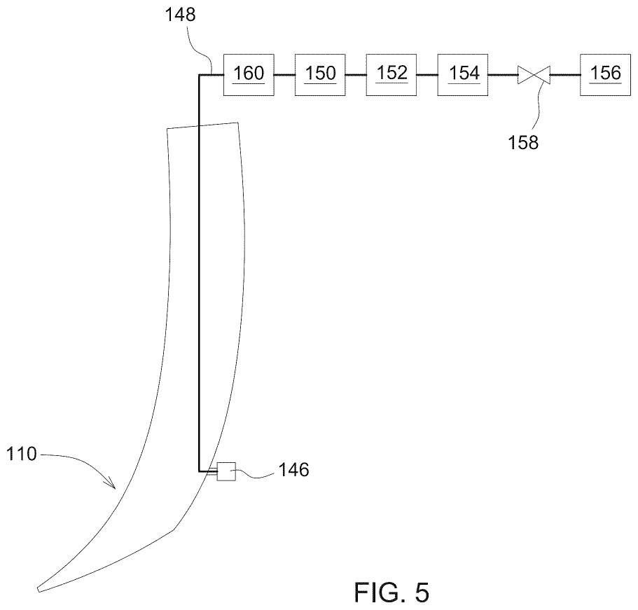

The at least one sensing probe 110 itself may be constructed in accordance with the teachings of the previously noted Herring, U.S. Patent Application Publ. No. 2023/0085819, the details of which are incorporated herein by reference. As is schematically shown in FIG. 5 the at least one sensing probe 110 may be structurally embodied as a part of any conventional tillage tool, such as a moldboard, chisel, disc, plow or the like. The specific nature of the associated tillage tool is not critical to operation of the system, and use of a tillage tool at all is not a requirement of the system. While one embodiment may utilize a cutting knife, positioned behind a coulter, other arrangements may also be used. More particularly, the combination of a working implement with the air intake of the system is beneficial in the sense that the operations of soil gas or other attribute measurement may take place simultaneously with plowing or other soil working applications to save time and expense.

The at least one sensing probe 110 may include an air intake 146 advantageously arranged to be placed into the soil environment being assessed and accept air emanating from that soil. More particularly, the air intake 146 is configured to facilitate rapid extraction of air from the soil in order to minimize intermixing of air extracted from the soil with atmospheric air above the soil, which can directly interfere with the accuracy of measurements taken by the at least one sensing probe 110.

The air intake 146 may be placed behind the trailing edge of the at least one sensing probe 110 so as to minimize the intake of soil particles. An air conduit 148 may communicate the air intake 146 with an air vacuum pump 150 to draw air in through the air intake 146. An air filter 160 may be provided between the air intake 146 and the air vacuum pump 150. In one embodiment the air vacuum pump 150 may be a high suction diaphragm pump producing a flow rate of approximately 8 L/min with a pressure range of approximately 500 to 8,000 mmHG. The soil gas sample drawn in through the air intake 146 is transferred to one or more sensors 152 for detecting a concentration of one or more soil gas constituents of the soil gas sampled by the soil gas sensor system. In one embodiment, the one or more sensors 152 include a CO₂ sensor 152A that detects and measures the amount of CO₂ that is present in the soil gas sample. Other sensors included in one or more sensors 152 may include a temperature sensor 152B and a relative humidity sensor 152C. An air flow sensor 154 may be included to detect plugging of the system.

In order to unblock a plugged line, a source of compressed air 156 may be communicated with the system via a valve 158, which under control of a controller 600 may periodically provide a reverse flow of air through the conduit 148 to blow any blockage out of the system. The details of the controller 600 are further described below with reference to FIG. 6 where it is seen that the controller 600 will be in communication with all of the aforementioned sensors.

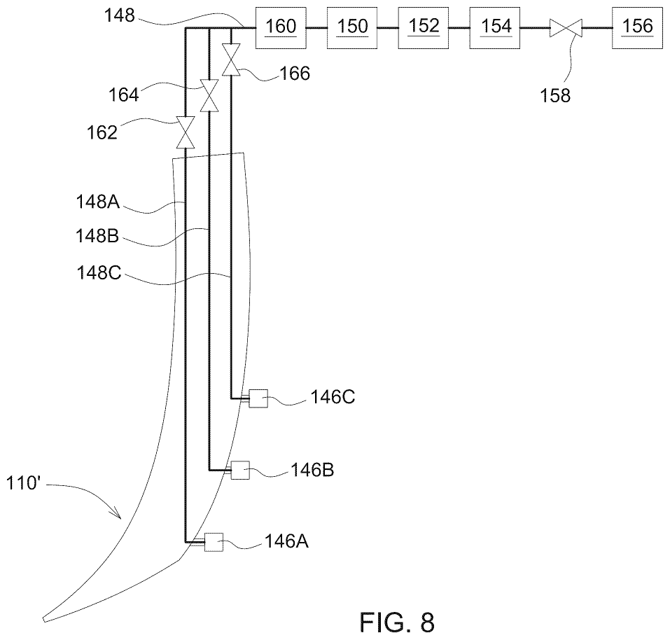

### Embodiment of FIGS. 7-8

A further embodiment of the implement 100 is shown in FIGS. 7 and 8 and is indicated as 100′. The soil gas sampling implement 100′ uses a modified sensing probe including a plurality of sensing probes arranged at different sensing depths. As schematically shown in FIG. 8 the sensing probe 110′ may again be structurally incorporated as part of any conventional tillage instrument, and may carry multiple air intakes 146A, 146B and 146C located at different distances from the implement frame 104.

Each of the air intakes 146A, 146B and 146C may be communicated with the air vacuum pump 150 via air conduits 148A, 148B and 148C, respectively, with the flow through a selected conduit being controlled by a bank of flow control valves 162, 164 and 166, respectively. The plurality of air intakes 146A, 146B and 146C may be alternatively described as a plurality of sensing probes, and it will be understood that the air intakes 146A, 146B and 146C can be structurally supported on one tillage tool as shown in FIG. 8 or could be located on separate tillage tools located side by side or one behind the other.

With the embodiment of FIGS. 7 and 8 the controller 600 can adjust the sensing depth 114 by sending a command signal to the appropriate valve 162, 164 and 166 to select one of the air intakes air intakes 146A, 146B and 146C for sampling, thereby adjusting the sensing depth.

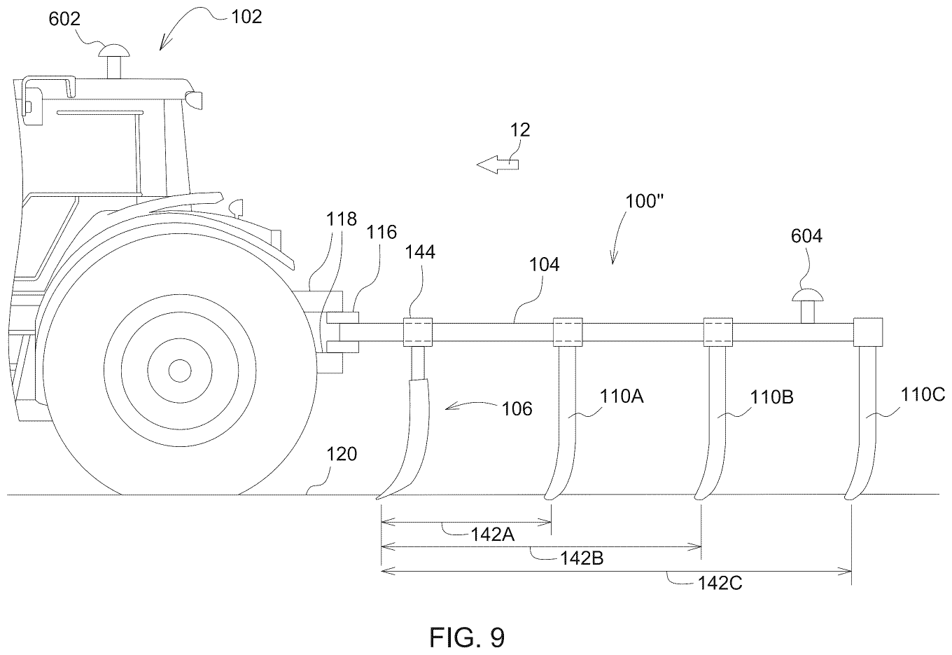

### Embodiment of FIG. 9

In another embodiment as seen in FIG. 9 and indicated as the soil gas sampling implement 100″, the at least one sensing probe 110 includes a plurality of sensing probes 110A, 110B and 110C arranged at different distances 142A, 142B and 142C behind the soil disrupting tool 106. With this embodiment the controller 600 is configured to receive the soil gas content signals from the plurality of sensing probes 110A, 110B and 110C and to correlate the signals with determined time delays between collection of samples by the plurality of sensing probes. This allows the controller to correlate the sensed gas concentration with elapsed time from the soil disruption by soil disrupting tool 106.

### Control System of FIG. 6

As schematically illustrated in FIG. 6, the soil gas sampling implement 100 includes a controller 600. The controller 600 may also be referred to as an automatic controller 600. The controller 600 may be part of the machine control system of the soil gas sampling implement 100, or it may be a separate control module. The controller 600 may be mounted in the operators'cab of the tractor 102 or it may be located on the implement 100 itself. The controller 600 may also be located remotely, especially in the case of the tractor 102 being autonomously controlled.

The controller 600 is configured to receive input signals from various sensors and operator inputs. For example, the controller 600 may receive GPS position signals from GPS receivers 602 carried by tractor 102 and/or GPS receivers 604 carried by implement 100. The GPS sensors 602 and/or 604 may generally be referred to as position sensor 602, 604 configured to generate a position signal 602S, 604S corresponding to a geographic location of the implement 100 and thus of the at least one sensing probe 110 within the field in which the implement 100 is working.

Controller 600 includes or may be associated with a processor 606, a computer readable medium 608, a data base 610 and an input/output module or control panel 612 having a display 614. An operator input 616 in the form of an input/output device, such as a keyboard, joystick or other user interface, is provided so that the human operator may input instructions to the controller. It is understood that the controller 600 described herein may be a single controller having all of the described functionality, or it may include multiple controllers wherein the described functionality is distributed among the multiple controllers.

Various operations, steps or algorithms as described in connection with the controller 600 can be embodied directly in hardware, in a computer program product 618 such as a software module executed by the processor 606, or in a combination of the two. The computer program product 618 can reside in RAM memory, flash memory, ROM memory, EPROM memory, EEPROM memory, registers, hard disk, a removable disk, or any other form of computer-readable medium 608 known in the art. An exemplary computer-readable medium 608 can be coupled to the processor 606 such that the processor can read information from, and write information to, the memory/storage medium. In the alternative, the medium can be integral to the processor. The processor and the medium can reside in an application specific integrated circuit (ASIC). The ASIC can reside in a user terminal. In the alternative, the processor and the medium can reside as discrete components in a user terminal.

The term “processor” as used herein may refer to at least general-purpose or specific-purpose processing devices and/or logic as may be understood by one of skill in the art, including but not limited to a microprocessor, a microcontroller, a state machine, and the like. A processor can also be implemented as a combination of computing devices, e.g., a combination of a DSP and a microprocessor, a plurality of microprocessors, one or more microprocessors in conjunction with a DSP core, or any other such configuration.

The controller 600 is functionally linked with the soil gas sensor system 108 and the position sensors 602, 604, for receiving the soil gas content signals 152S and the position signals 602S, 604S. The controller 600 is configured to send a command signal to the soil gas sensor system 108 to adjust the sensing depth 114 at which the soil gas is sampled as the implement 100 traverses the field.

In the embodiment of FIGS. 1-5 the command signal may be a command signal 126C directed to the actuator 126, 128 to direct movement of the piston 126 to adjust the sensing depth 114 of the at least one sensing probe 110 and particularly of the air intake 146.

In the embodiment of FIGS. 7-8 the command signal may be a command signal 162C, 164C or 166C to open the appropriate valve 162, 164 or 166 to draw in a gas sample from the selected air intake 146A, 146B or 146C, respectively.

The system disclosed herein which provides for variable control of sensing depth 114 as well as sampling at variable time delays after initial soil disruption by soil disrupting tool 106 allows the controller 600 to do sophisticated analysis of the constituent gases of the soil gas from the field being worked, and this can be done simultaneously with the actual tillage of the field for traditional agricultural purposes. Such analysis will allow the manager of the field to make informed decisions regarding addition of soil supplements.

Not only can the controller 600 adjust the sensing depth 114 in the various manners described, but the controller 600 can be configured to adjust the sensing depth 114 in a repeating pattern as the implement 100 traverses a field. The repeating pattern may for example be a sawtooth pattern or a sinusoidal pattern where the sensing depth 114 repeatedly varies between a deeper penetration and a shallower penetration as the implement 100 traverses the field.

Also, the controller 600 may be configured to adjust the sensing depth 114 at least in part on a detected value of another soil attribute. The other soil attribute may for example be soil moisture as detected by sensor 152C or soil temperature as detected by sensor 152B.

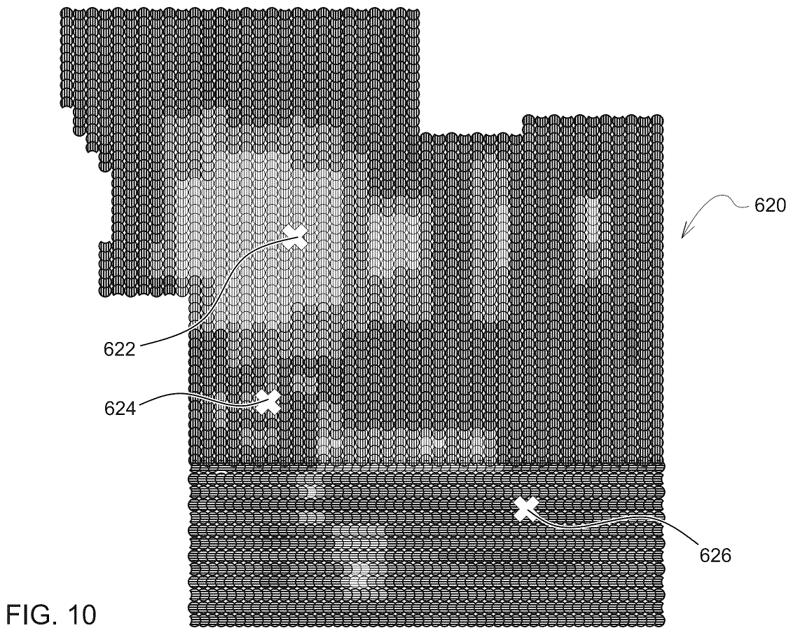

The controller 600 may be further configured to map the soil gas content signal or other parameter derived from the soil gas content signal relative to the geographic position of the implement within the field. Such a map 620 is schematically shown in FIG. 10. The measured CO₂ efflux may for example be shown by shading or color gradations on the map 620. This data may then be used to manage the treatment of the field.

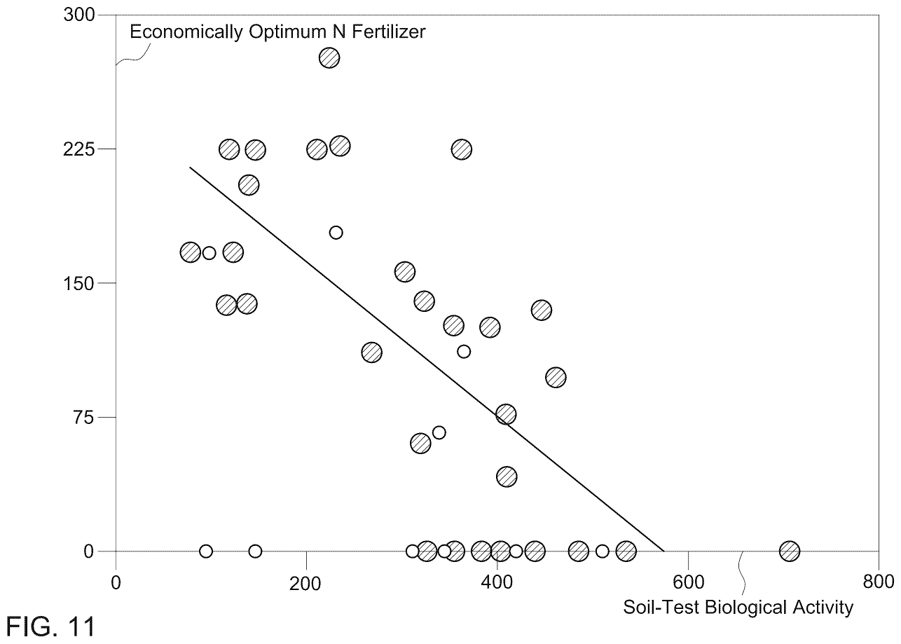

For example, the academic literature shows a quantifiable relationship between an economically optimum nitrogen application rate to a field, and the soil CO₂ efflux from the field. Such relationships may take the form generally shown in FIG. 11 where the economically optimum nitrogen fertilizer application rate is shown on the vertical axis and a measure of biological activity in the soil, as indicated by CO₂ efflux, is shown on the horizontal axis.

Furthermore, by correlating the CO₂ concentration data gathered with the at least one sensing probe 110 of the present disclosure, with other data gathered from static probes within the field even greater insights may be obtained. FIG. 10 illustrates three static sensor locations 622, 624 and 626 selectively located at locations of expected high, medium and low CO₂ efflux within the field.

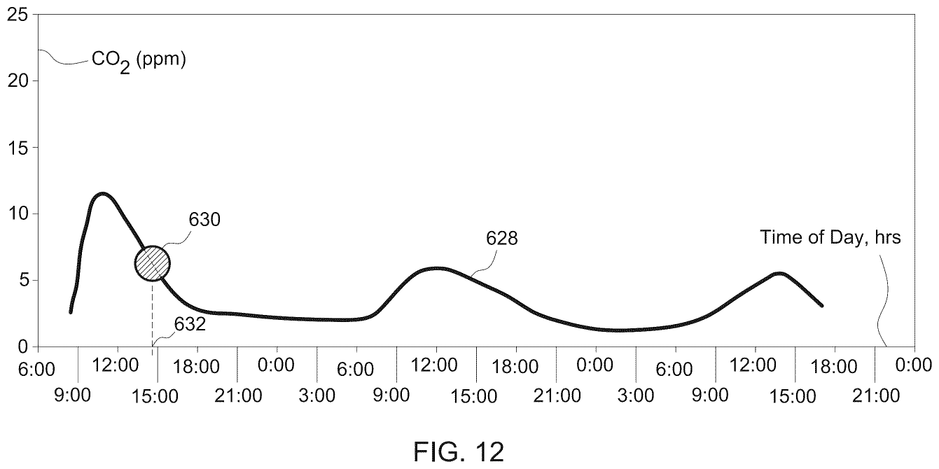

FIG. 12 graphically illustrates how the data from the static sensors may be utilized to give greater insight into the significance of the data gathered by the mobile sensors 110. The curve 628 may represent the CO₂ concentration detected by one or more of the static sensor locations 622, 624 and 626 as a function of time of day. The dot 630 may represent one measure of CO₂ concentration made by the at least one sensing probe 110 at time 632. By looking at the shape of the curve 628 at time 632 it is apparent that the CO₂ efflux of the field is rapidly dropping as a function of time, due to some environmental factor, and this can be taken into account when evaluating the overall CO₂ efflux data for the field and making a determination as to whether and where in the field it is desirable to add further nitrogen fertilizer and in what amounts to add the nitrogen fertilizer.

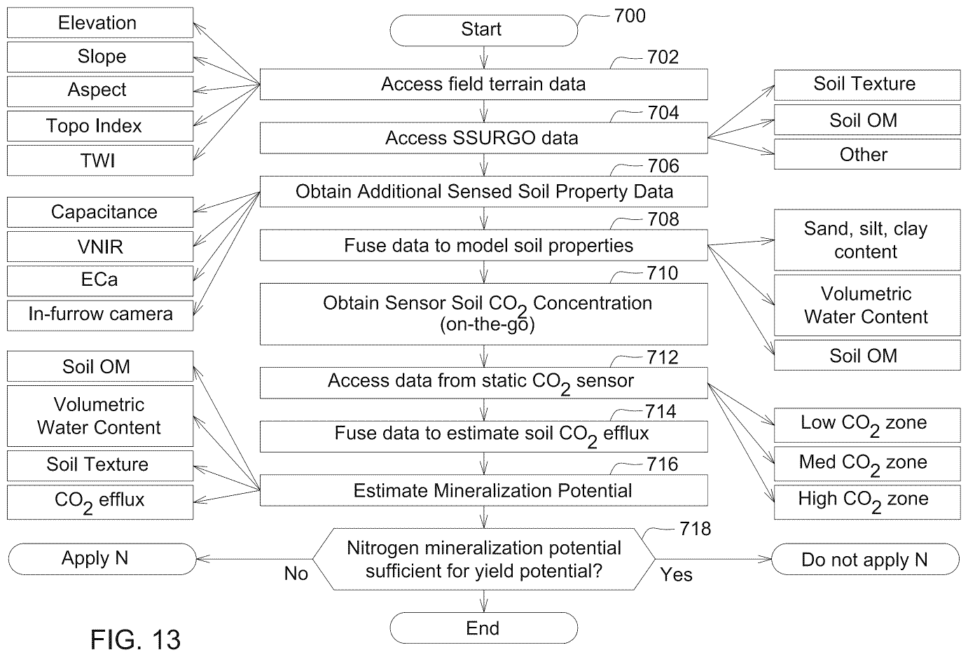

FIG. 13 is a flow chart of one configuration of the controller 600 for analysis of the soil gas concentration signal 152S in combination with other information including detected values of other soil attributes to estimate CO₂ efflux.

The process starts at block 700 and then at block 702 accesses various field terrain data such as elevation, slope, aspect, topo index and TWI, which data may be stored as a function of geographic location. Next at block 704 SSURGO data from the Soil Survey Geographic Database may be accessed to determine soil texture, soil OM, or other attributes. Next at block 706 the controller 600 may obtain additional sensed soil property data such as capacitance, VNIR, ECa or in-furrow camera data. At block 708 the controller 600 may fuse all of the available data to model the soil properties of the field, such as sand, silt and clay content, volumetric water content, and soil OM.

At block 710 the controller 600 may obtain the on-the-go sensor data from the at least one sensing probe 110 using any of the embodiments of the implement 100 disclosed above to measure CO₂ concentration.

At block 712 the controller 600 may access data from the static sensors such as 620, 622 and 624 shown in FIG. 11. At block 714 the controller 600 may fuse the on-the-go and static sensor data to estimate soil CO₂ efflux throughout the field. At block 716 the controller 600 may estimate the mineralization potential of the field based on soil OM, volumetric water content, soil texture, CO₂ efflux and any other relevant factors.

At block 718 a decision is made as to whether the nitrogen mineralization potential of the field is sufficient to achieve the yield potential of the field. If the answer is “yes” then there is no need to add further nitrogen fertilizer. If the answer is “no” then the decision may be made to add further nitrogen fertilizer and the appropriate quantities of nitrogen fertilizer to be added may be quantified.

The process described may be characterized as analyzing the soil gas concentration signal in combination with a detected value of at least one other soil attribute to estimate carbon dioxide efflux, the at least one other soil attribute being selected from the group consisting of: soil moisture; soil temperature; measurements from a static carbon dioxide sensor; historical field data; field terrain data; and soil property data.

Thus, it is seen that the apparatus and methods of the embodiments disclosed herein readily achieve the ends and advantages mentioned as well as those inherent therein. While certain preferred embodiments have been illustrated and described for purposes of the present disclosure, numerous changes in the arrangement and construction of parts and steps may be made by those skilled in the art, which changes are encompassed within the scope and spirit of the present invention as defined by the appended claims.

What is claimed is:

1. A soil gas sampling implement, comprising:
a soil gas sensor system configured to sample soil gas at multiple depths from soil of a field being traversed by the implement and configured to generate a soil gas content signal corresponding to a concentration of one or more soil gas constituents of the soil gas sampled by the soil gas sensor system, the soil gas sensor system including at least one sensing probe configured to collect soil gas samples;
a position sensor configured to generate a position signal corresponding to a geographic location of the implement within the field; and
a controller functionally linked with the soil gas sensor system and the position sensor for receiving the soil gas content signal and the position signal, the controller being configured to send a command signal to the soil gas sensor system to adjust a sensing depth at which the soil gas is sampled as the implement traverses the field.

2. The soil gas sampling implement of claim 1, further comprising:
an implement frame; and
a depth adjustment assembly adjustably supporting the at least one sensing probe from the implement frame so that a sensing depth of the at least one sensing probe within the soil is adjustable;
wherein the controller is further configured to send the command signal to the depth adjustment assembly to adjust the sensing depth of the at least one sensing probe.

3. The soil gas sampling implement of claim 1, wherein:
the at least one sensing probe includes a plurality of sensing probes arranged at different sensing depths; and
wherein the controller is configured to send the command signal to select a selected one of the sensing probes to adjust the sensing depth.

4. The soil gas sampling implement of claim 1, further comprising:
a soil disrupting tool supported from the implement in front of the at least one sensing probe with respect to a forward direction of travel

5. The soil gas sampling implement of claim 4, wherein:
the soil disrupting tool is spaced from the at least one sensing probe by a distance in a range of from 1 to 12 feet in the direction of travel so as to create a time delay between disruption of the soil by the soil disrupting tool and sampling of the soil gas by the at least one sensing probe.

6. The soil gas sampling implement of claim 4, further comprising:
an implement frame;
wherein one or both of the soil disrupting tool and the at least one sensing probe is adjustably mounted relative to the implement frame so that a distance between the soil disrupting tool and the sensing probe is adjustable.

7. The soil gas sampling implement of claim 4, wherein:
the at least one sensing probe includes a plurality of sensing probes arranged at different distances behind the soil disrupting tool; and
wherein the controller is configured to receive the soil gas content signals from the plurality of sensing probes and to correlate the signals with determined time delays between collection of samples by the plurality of sensing probes.

8. The soil gas sampling implement of claim 1, wherein:
the controller is configured to adjust the sensing depth of the sensing probe in a repeating pattern as the implement traverses the field.

9. The soil gas sampling implement of claim 1, wherein:
the controller is configured to adjust the sensing depth at which soil gas is sampled based at least in part on a detected value of another soil attribute.

10. The soil gas sampling implement of claim 9, wherein:
the other soil attribute is selected from the group consisting of soil moisture and soil temperature.

11. The soil gas sampling implement of claim 1, wherein:
the controller is configured to map the soil gas content signal or other parameter derived from the soil gas content signal relative to the geographic position of the implement within the field.

12. The soil gas sampling implement of claim 1, wherein:
the one or more soil gas constituents measured by the soil gas sensor system includes carbon dioxide.

13. A method of sampling soil gas, comprising:
sampling soil gas from soil of a field with at least one sensing probe of an implement as the field is being traversed by the implement;
generating a soil gas concentration signal corresponding to a concentration of one or more soil gas constituents of the soil gas sampled by the at least one sensing probe;
detecting a geographic location of the implement within the field using a position sensor associated with the implement and configured to generate a position signal corresponding to a geographic location of the implement within the field;
receiving the soil gas concentration signal and the position signal with a controller; and
generating a command signal with the controller and thereby adjusting a sensing depth at which the soil gas is sampled as the implement traverses the field.

14. The method of claim 13, further comprising:
disrupting the soil ahead of the at least one sensing probe with a soil disrupting tool supported in front of the sensing probe with respect to a forward direction of travel.

15. The method of claim 13, wherein:
the adjusting of the sensing depth includes adjusting the sensing depth of the sensing probe in a repeating pattern as the implement traverses the field.

16. The method of claim 13, wherein:
the adjusting of the sensing depth is based at least in part on a detected value of another soil attribute.

17. The method of claim 16, wherein:
the other soil attribute is selected from the group consisting of soil moisture and soil temperature.

18. The method of claim 13, further comprising:
mapping with the controller the soil gas concentration signal or other parameter derived from the soil gas concentration signal relative to the geographic position of the implement within the field.

19. The method of claim 13, wherein:
the one or more soil gas constituents of the soil gas sampled by the sensing probe and corresponding to the soil gas concentration signal includes carbon dioxide.

20. The method of claim 13, further comprising:
analyzing the soil gas concentration signal in combination with a detected value of at least one other soil attribute to estimate carbon dioxide efflux, the at least one other soil attribute being selected from the group consisting of:
soil moisture;
soil temperature;
measurements from a static carbon dioxide sensor;
historical field data;
field terrain data; and
soil property data.
